(19) United States
(12) Patent Application Publication
(10) Pub. No.: US 2011/0045086 A1
(43) Pub. Date: Feb. 24, 2011

(54) Steroid Nebuliser Formulation

(75) Inventors: Ian Gardner Cameron McAffer, Stevanage (GB); Peter Ernest Tasko, Stevanage (GB); Graham John Swift, Stevanage (GB); Susheela Giafrancesco, Stevanage (GB)

Correspondence Address: Florek & Endres PLLC, 1156 Avenue of the Americas, Suite 600, New York, NY, 10036, US

(73) Assignee: BREATH LTD, Tatsfield, Westerham (GB)

(21) Appl. No.: 12/988,048

(22) PCT Filed: Apr. 16, 2009
(86) PCT No.: PCT/GB09/00989

§ 371 Date: Nov. 9, 2010

(30) Foreign Application Priority Data
Apr. 16, 2008 (GB) 0806873.6

(51) Int. Cl. A61K 9/14; A61K 31/573; A61P 11/00; B32B 5/16

(52) U.S. Cl. 424/489; 514/180; 428/402

(57) ABSTRACT

A nebulizer formulation comprises particles of size 0.5-3 microns obtained by crystallization of beclomethasone diproprionate monohydrate in the presence of ultrasound.

### FIELD

The present invention relates to nebuliser formulations of steroids, in particular nebuliser formulations of beclomethasone. Preferred formulations of the invention comprise beclomethasone hydrate.

### BACKGROUND

Delivery of steroids to the lungs via aerosol is widely known and used for the treatment of a number of disease, including asthma, airways disease and chronic obstructive pulmonary disease (COPD). Formulations are generally administered via dry powder inhaler (dpi), metered dose inhaler (mdi) and, to a lesser extent, nebuliser.

### PROBLEMS

A nebuliser formulation of beclomethasone is known, containing a suspension of beclomethasone diproprionate particles falling in the size range 2-5 microns. This formulation is successfully used for administration of beclomethasone, with apparently suitable particle size and particle size distribution. However, the inventors hereof have identified a problem with the formulation, namely that it does not store well, any period of storage tending to result in product settling in the container, e.g. in the bottom of the ampoule or if inverted in the head, and being difficult to resuspend even after significant agitation; this resuspension is, to the inventors, notably more difficult than with other steroids used in nebuliser formulations. In addition, the effective dose of beclomethasone at point of delivery to the patient is rather lower than that contained in the formulation, it being acknowledged that a certain loss of product occurs during delivery, though again, to the inventors, the amount lost is higher than for other steroids. This loss is compensated in the amount of active included in the formulation, a solution regarded as acceptable. Nevertheless, it would be desirable to reduce this loss: any loss is to some degree uncontrollable and hence affects the reliability of dosing.

A method of preparing small crystals is described in WO 02/089942, in which crystallization occurs in the presence of ultrasound. WO 2004/073827 describes preparation of aerosol formulation for mdi and dpi uses, again using ultrasound during crystallization of the active component.

An aim of the present invention is to provide alternative, preferably improved beclomethasone nebuliser formulations and methods of making the same, in particular, beclomethasone formulations that are easier to resuspend and/or exhibit less loss of product in use between the ampoule and the point of delivery.

The present invention is based upon the realisation by the inventors of specific difficulties in preparation and use of beclomethasone-containing nebuliser formulations.

### INVENTION

Accordingly, the invention provides a nebuliser formulation, comprising beclomethasone particles of size 0.5-10 microns obtained by crystallization of beclomethasone in the presence of ultrasound. The present invention also provides a method of preparing a nebulizer formulation of beclomethasone, comprising combining (i) beclomethasone particles crystallized in the presence of ultrasound, with (ii) a pharmaceutically acceptable carrier. Preferably the beclomethasone is beclomethasone hydrate, preferably beclomethasone monohydrate. Beclomethasone dipropionate monohydrate and beclomethasone valerate monohydrate are particularly suitable for use in formulations of the invention.

The use of ultrasound is found to yield a formulation which does not so easily settle or form a plug in storage or, if it does, can be resuspended more readily. In addition, initial indications are that less product is lost in nebuliser equipment and so for a given does in the ampoule a higher dose reaches the patient. Dosing can be hence more reliable.

Preferred formulations comprise a suspension of beclomethasone particles of size 2-5 microns, more preferably 2-3 microns. Further, it is preferred that a substantial proportion of the product be within these stated size ranges so that a substantial proportion will reach the patient's lungs, and preferably at least 75%, more preferably at least 90% of the beclomethasone is within the stated size range. It is further preferred that 100% of particles (by number) are 10 microns or less in diameter, 95% or more are 5 microns or less in diameter and 80% or more are 3 microns or less in diameter. It is particularly preferred that 80% or more of the particles are from 2 to 3 microns in diameter.

Particularly preferred formulations of the invention comprise a suspension of beclomethasone hydrate particles of size 0.5-5 microns, more preferably 0.5-3 microns. Further, it is preferred that a substantial proportion of the product be within these stated size ranges so that a substantial proportion will reach the patient's lungs, and preferably at least 75%, more preferably at least 80% of the beclomethasone hydrate is within the stated size range. It is further preferred that 100% of particles (by number) are 10 microns or less in diameter, 95% or more are 5 microns or less in diameter and 80% or more are 3 microns or less in diameter. It is particularly preferred that 80% or more of the particles are from 0.5 to 3 microns in diameter.

Particle size or particle diameter as used herein can be suitably determined by laser diffraction based methods, for example as described in ISO Standard 13320-1. Laser diffraction particle sizing apparatus such as the Malvern Mastersizer 2000™ can be used.

The amount of beclomethasone should be sufficient to provide an effective dose to the patient, dependent upon anticipated nebulising time and patient age, weight and disease state. The nebulizer formulations of the invention comprise a suspension of crystalline beclomethasone particles in an aqueous carrier, for use in known nebulizing apparatus. Typically, formulations of the invention comprise 0.4 mg-0.8 mg beclomethasone and a surfactant in 1 ml-3 ml of a pharmaceutically acceptable carrier, especially about 2 ml. Particular embodiments contain beclomethasone in a formulation of about 1 ml. A specific embodiment contains about 0.8 mg beclomethasone in a formulation of about 1 ml. The formulations are preferably sterile and may further comprise sodium chloride and/or a buffer.

Preferably, formulations of the invention comprise 0.4 mg-0.8 mg beclomethasone hydrate and a surfactant in 1 ml-3 ml of a pharmaceutically acceptable carrier, especially about 2 ml. Particular embodiments contain beclomethasone hydrate in a formulation of about 1 ml. The formulations are preferably sterile and may further comprise sodium chloride and/or a buffer.

A specific embodiment of the invention provides a sterile nebulizer formulation, comprising beclomethasone particles of size 2-3 microns obtained by crystallization of beclomethasone in the presence of ultrasound, wherein the formulation comprises 0.4 mg-0.8 mg of beclomethasone, a surfactant, sodium chloride and, optionally, a buffer in 1 ml-3 ml of a pharmaceutically acceptable carrier.

A further specific embodiment of the invention provides a sterile nebulizer formulation, comprising beclomethasone hydrate particles of size 2-3 microns obtained by crystallization of beclomethasone in the presence of ultrasound, wherein the formulation comprises 0.4 mg-0.8 mg of beclomethasone hydrate, a surfactant, sodium chloride and, optionally, a buffer in 1 ml-3 ml of a pharmaceutically acceptable carrier.

In use of the invention, beclomethasone is obtained which is not needle-shaped as hitherto known but forms more rounder-edged, spherical particles. Beclomethasone hydrate particles obtained by the invention are characterized by a regular shape and smooth surface morphology. As mentioned, these tend not to settle the way the existing formulations do, but if the preparation of beclomethasone obtained using this method does settle then it is found to be easily resuspended.

Beclomethasone is suitably crystallized by forming a solution of beclomethasone in a solvent, forming a suspension of droplets of the solution in a non-solvent of beclomethasone, and applying ultrasound to the droplets. The beclomethasone in the suspended droplets, which may be mainly or entirely beclomethasone, crystallizes to form particles of a generally spherical type. More specifically, it is crystallized by dissolving it in a solvent, forming droplets of the solution, for example by generating an aerosol from this solution, forming a dispersion of the droplets in a non-solvent of beclomethasone and subjecting the droplets to ultrasound to initiate or effect crystallization of the beclomethasone.

When water is used as the non-solvent beclomethasone hydrate is formed and when crystallization is initiated or effected by ultrasound regular shaped particles with a generally smooth surface morphology are formed. These regular shaped particles can be rounder-edged and more spherical. Other shapes may also be formed.

Droplets can be prepared by electrohydrodynamic spraying, atomizing using high pressure, spray nozzles, nebulisers, transducers such as piezoelectric transducers or ultrasonic transducers or other aerosol generators.

To obtain the desired particle size of the crystalline beclomethasone the size of the droplets and the amount of beclomethasone in the solvent are varied and controlled. The process is to a certain extent empirical as different systems operating under similar conditions will produce different end particle sizes. However, the droplets should generally be micron sized, say in the range 1-100 microns, preferably 3-30 microns to yield crystals in the size range 0.5-10 microns.

To obtain more generally spherical crystals it is preferred that the droplets of solvent contain a high proportion of beclomethasone. Solvent evaporates from the solvent droplets in the aerosol and this can be controlled and optimized so that the droplets when they are collected in or combined with the beclomethasone non-solvent contain at least 80%, more preferably at least 90%, more preferably at least 95% beclomethasone by weight of droplet.

Hence by variation of a number of parameters, including % product in the droplets and droplet size, the ultimate crystal particle size can be controlled so that particles within the ranges 0.5-10 microns, preferably 2-5 microns, more preferably 2-3 microns are obtained; it being particularly preferred that 80% of the particles by number are from 2 microns to 3 microns in diameter.

Suitable solvents for beclomethasone are alcohols and ketones, in particular low molecular weight ketones, alcohols and halogenated alkanes, specific examples being acetone, ethanol, methanol and dichloromethane.

The non-solvent should dissolve a very low amount of the beclomethasone, preferably not more than 0.1% w/w; it may be miscible with the solvent and an emulsifier or other agent may be added to aid stability of the droplets suspension. Suitable non-solvents for beclomethasone are water and mixtures of water, ketones and/or alcohols.

The method thus includes forming a suspension of (i) droplets containing beclomethasone dissolved in a solvent, in (ii) a non-solvent of beclomethasone, and applying ultrasound to the droplets. Ultrasound is applied to and causes crystallization of beclomethasone in the droplets. The method may comprise crystallizing the beclomethasone in the pharmaceutically acceptable carrier, or forming drug crystals and the combining these with the carrier.

A specific embodiment of the method includes forming a suspension of (i) droplets containing beclomethasone dissolved in a solvent, in (ii) water, and applying ultrasound to the suspension. Ultrasound is applied to and causes crystallization of beclomethasone hydrate in the suspension. The method may comprise crystallizing the beclomethasone hydrate in a pharmaceutically acceptable carrier, or forming drug crystals and the combining these with the carrier.

Crystallization is effected or initiated by applying ultrasound to the beclomethasone. Crystallization is also effected or initiated by applying ultrasound to the beclomethasone hydrate. The ultrasound may be applied continuously or discontinuously such as in a pulsed manner. It may be applied using a variety or devices, such as a probe inserted into the suspension.

Whilst the frequency and amplitude may vary, beclomethasone may be crystallized in the presence of ultrasound having frequency from 20 kHz to 5 MHz. Separately, ultrasound may have an intensity of 0.2 W/cm² or higher, or 0.3 W/cm² or higher.

Particularly preferred embodiments of the invention produce nebuliser formulations for use in treatment of asthma or COPD. A method for making such formulations comprises combining ultrasound-crystallized beclomethasone particles with a surfactant, under sterile conditions, to obtain a sterile nebulizer formulation of a volume from 1-3 ml. Specific formulations contain about 2 ml of formulation.

A further preferred method for making such formulations comprises combining ultrasound-crystallized beclomethasone hydrate particles with a surfactant, under sterile conditions, to obtain a sterile nebulizer formulation of a volume from 1-3 ml. Specific formulations contain about 2 ml of formulation.

Reference herein to beclomethasone is reference to the drug substance in any of its suitable and available forms, including salts and other derivatives thereof, such as but not limited to beclomethasone dipropionate and beclomethasone valerate, etc.

### EXAMPLES

### Example 1

### Beclomethasone Formulation

A beclomethasone nebulizer formulation is prepared, by dissolving beclomethasone in ethanol and then forming a suspension of the beclomethasone solution in water, and crystallizing the beclomethasone by application of ultrasound, as described in WO 2004/073827.

The operating parameters including flow rate and ultrasound power are varied so as to obtain a particle size for crystallized beclomethasone substantially within the size range 2-3 microns.

The beclomethasone hydrate obtained is formulated with surfactant and carriers, then subjected to end-sterilization by irradiation to yield the end formulations having composition:—

1

| | |
|---|---|
| Beclomethasone | 0.4 mg |
| Polysorbate 20 | 2.0 g |
| Sodium Chloride | 18.0 g |
| Sorbitan monolaurate | 0.4 g |
| Sterile water | qs, 2 ml total volume |

2

| | |
|---|---|
| Beclomethasone | 0.8 mg |
| Polysorbate 80 | 2.0 g |
| Sorbitan monolaurate | 18.0 g |
| Sorbitan monolaurate | 0.4 g |
| Sodium Chloride | 18.0 g |
| Sterile water | qs, 2 ml total volume |

3

| | |
|---|---|
| Beclomethasone | 0.4 mg |
| Polysorbate 20 | 2.0 g |
| Sodium Chloride | 9.0 g |
| Sorbitan monolaurate | 0.4 g |
| Sterile water | qs, 1 ml total volume |

4

| | |
|---|---|
| Beclomethasone | 0.8 mg |
| Polysorbate 80 | 2.0 g |
| Sorbitan monolaurate | 18.0 g |
| Sorbitan monolaurate | 0.4 g |
| Sodium Chloride | 9.0 g |
| Sterile water | qs, 1 ml total volume |

### Example 2

Beclomethasone was crystallized utilizing SAX+™ processing, as provided by Prosonix Ltd., Oxford, UK. Briefly, this method comprised formation of a drug substance solution followed by its atomization, controlled evaporation of the solvent, collection of the pre-concentrated viscous droplets in a vessel containing non-solvent and crystallisation via nucleation with power ultrasound. The product slurry was then transferred to solid isolation, by spray-drying or supercritical carbon dioxide drying. Further details of this method are as described in WO 2004/073827.

Beclomethasone hydrate obtained by SAX+™ processing

### Protocol:

Input: 6 g of anhydrous beclomethasone diproprionate (BDP)
3% w/v solution of anhydrous BDP in methanol was atomized and sonoprocessed in water
Temperature: 0° C.
Particles were isolated by spray drying

Differential scanning calorimetry (DSC) and TGA following isolation by spray drying showed highly crystalline BDP hydrate.

SEM showed particles with smooth surfaces and homogeneous morphology. Dry Sympatec PSD analysis confirmed that the particle size distribution was well within the inhalation range.

Table 1 shows the results of dry Sympatec PSD analysis:

| Cumulative distribution Q3 (%) | Particle Size (μ) |
|---|---|
| X₁₀ | 0.51 |
| X₅₀ | 1.35 |
| X₉₀ | 3.17 |

In order to evaluate the effect of humidity on prolonged storage processed BDP hydrate was subjected to 20% relative humidity (RH) for 48 hours.

DVS mass plot of the processed BDP hydrate showed that during storage the sample initially underwent considerable weight loss due to partial dehydration. The sample achieved a steady state after about 1500 minutes. The loss of water from the sample is likely to reflect the loss of free water remaining in the sample after spray drying, as this drying technique is usually not 100% efficient.

These results indicate that BDP formed a hydrate at a very low moisture content, and is anticipated to retain stability on prolonged storage.

The sample recovered after storage was analysed by DSC, TGA, PSD and SEM.

The DSC trace of the stored sample indicated no variation in the thermal behavior of the sample post-humidity treatment. The hydrated sample exhibited higher stability on prolonged storage than anhydrous BDP (see comparative example 3).

PSD showed no significant variation of particle size and SEM analysis showed identical morphology to the pre-storage sample.

Table 2 shows the results of dry Sympatec PSD analysis of the post-storage sample:

| Cumulative distribution Q3 (%) | Particle Size (μ) |
|---|---|
| X₁₀ | 0.51 |
| X₅₀ | 1.37 |
| X₉₀ | 2.95 |

### COMPARATIVE EXAMPLES

SAX+™ processing was carried out as described in Example 2 above.

### Comparative Example 1

SAX+™ processing with n-heptane

### Protocol:

Input: 2 g of anhydrous beclomethasone diproprionate (BDP)
1.25% w/v solution of (1:4-SS:BDP) in methanol was atomized into n-heptane
Temperature: 0° C.
Particles were isolated by supercritical CO₂

Microscope imaging of the suspension prior to isolation showed partially agglomerated particles up to 5 μm.

Differential scanning calorimetry (DSC) following isolation by supercritical CO₂ showed evidence for highly crystalline anhydrous BDP at least as pure as the supplied material. The results confirmed that isolation by supercritical CO₂ extraction effected the desolvation of the BDP n-heptane solvate.

Microscope imaging and SEM showed that significant crystal growth occurred during the supercritical CO₂ isolation with particles of up to 100 μm being observed. This was confirmed by dry Sympatec PSD analysis which showed bimodal distribution and particles up to 150 μm.

Table 3 shows the results of dry Sympatec PSD analysis:

| Cumulative distribution Q3 (%) | Particle Size (μ) |
|---|---|
| X₁₀ | 1.26 |
| X₅₀ | 12.58 |
| X₉₀ | 61.27 |

### Comparative Example 2

SAX+™ processing with cyclopentane

### Protocol:

Input: 2 g of anhydrous beclomethasone diproprionate (BDP)
1.25% w/v solution of (1:4-SS:BDP) in methanol was atomized into cyclopentane
Temperature: 0° C.
Particles were isolated by supercritical CO₂

Microscope imaging of the suspension prior to isolation showed partially agglomerated particles up to 5 μm.

Differential scanning calorimetry (DSC) following isolation by supercritical CO₂ showed evidence for amorphous BDP but no evidence for a BDP solvate.

SEM showed particles with smooth surfaces and homogeneous morphology but also large clusters up to 10 μm in size. Dry Sympatec PSD analysis confirmed the presence of mostly ˜4 μm particles alongside larger clusters or agglomerates larger than 20 μm.

Table 4 shows the results of dry Sympatec PSD analysis:

| Cumulative distribution Q3 (%) | Particle Size (μ) |
|---|---|
| X₁₀ | 0.84 |
| X₅₀ | 3.58 |
| X₉₀ | 8.76 |

### Comparative Example 3

### Storage Stability of Anhydrous BDP

In order to evaluate the effect of humidity on prolonged storage anhydrous BDP was subjected to 20% relative humidity (RH) for 48 hours. The pre-storage sample was characterized by TGA, DSC, PSD and SEM analysis.

The pre-storage material was shown to be anhydrous and highly crystalline.

Table 5 shows the results of dry Sympatec PSD analysis of the pre-storage sample:

| Cumulative distribution Q3 (%) | Particle Size (μ) |
|---|---|
| X₁₀ | 0.43 |
| X₅₀ | 1.10 |
| X₉₀ | 2.24 |

DVS mass plot of the anhydrous BDP during storage indicated considerable weight changes, possibly attributable to transition of anhydrous material in the presence of moisture.

The sample recovered after storage was analyzed by DSC, PSD and SEM.

The DSC trace of the stored sample indicated formation of BDP hydrate, with the post-storage sample exhibiting a significant endothermic event prior to the anhydrous BDP melt event, indicating instability in storage.

PSD showed no significant variation of particle size and SEM analysis showed no significant changes in morphology.

Table 6 shows the results of dry Sympatec PSD analysis of the post-storage sample:

| Cumulative distribution Q3 (%) | Particle Size (μ) |
|---|---|
| X₁₀ | 0.42 |
| X₅₀ | 1.07 |
| X₉₀ | 2.10 |

The invention thus provides beclomethasone-containing nebuliser formulations and manufacture thereof.

1. A nebuliser formulation, comprising crystallized beclomethasone hydrate particles of size 0.5-10 microns, wherein the particles are obtainable by forming a solution of beclomethasone in a solvent, forming droplets of the solution by generating an aerosol from this solution, forming a dispersion of the droplets in water and applying ultrasound to the droplets so as to initiate or effect crystallization of the beclomethasone.

2. A formulation according to claim 1, comprising beclomethasone particles of size 0.5-5 microns.

3. A formulation according to claim 1, comprising beclomethasone particles of size 0.5-3 microns.

4. A formulation according to claim 1, wherein at least 50% of the beclomethasone particles are of size 0.5-3 microns.

5. A formulation according to claim 4, wherein at least 75% of the beclomethasone particles are of size 0.5-3 microns.

6. A formulation according to claim 5, wherein at least 80% of the beclomethasone particles are of size 0.5-3 microns.

7. A formulation according to claim 1, wherein the beclomethasone hydrate is beclomethasone monohydrate.

8. A formulation according to claim 7, wherein the beclomethasone hydrate is beclomethasone diproprionate.

9. A formulation according to claim 1, wherein beclomethasone is crystallized in the presence of ultrasound having frequency from 20 kHz to 5 MHz.

10. A formulation according to claim 1, wherein beclomethasone is crystallized in the presence of ultrasound having an intensity of 0.2 W/cm² or higher.

11. A formulation according to claim 10, wherein beclomethasone is crystallized in the presence of ultrasound having an intensity of 0.3 W/cm² or higher.

12. A formulation according to claim 1, comprising 0.4 mg-0.8 mg beclomethasone and a surfactant in 1 ml-3 ml of a pharmaceutically acceptable carrier.

13. A formulation according to claim 12, wherein the formulation is sterile and further comprises sodium chloride and/or a buffer.

14. A sterile nebulizer formulation, comprising beclomethasone hydrate particles of size 2-3 microns obtained by forming a solution of beclomethasone in a solvent, forming droplets of the solution by generating an aerosol from this solution, forming a dispersion of the droplets in water and applying ultrasound to the droplets so as to initiate or effect crystallization of the beclomethasone, wherein the formulation comprises 0.4 mg-0.8 mg of beclomethasone, a surfactant, sodium chloride and, optionally, a buffer in 1 ml-3 ml of a pharmaceutically acceptable carrier.

15. A method of preparing a nebulizer formulation of beclomethasone hydrate, comprising combining (I) beclomethasone hydrate particles crystallized in the presence of ultrasound, with (II) a pharmaceutically acceptable carrier wherein the particles are obtained by forming a solution of beclomethasone in a solvent, forming droplets of the solution by generating an aerosol from this solution, forming a dispersion of the droplets in water and applying ultrasound to the droplets so as to initiate or effect crystallization of the beclomethasone.

16. A method according to claim 15, wherein the droplets are 1 to 100 microns in size.

17. A method according to claim 16, wherein the droplets are 3 to 30 microns in size.

18. A method according to claim 15, wherein the solvent is evapourated from the droplets in the aerosol so that when the droplets are collected in or combined with the water they contain at least 80% beclomethasone by weight of droplet.

19. A method according to claim 15, comprising crystallizing beclomethasone in ultrasound of frequency from 20 kHz to 5 MHz.

20. A method according to claim 15, comprising crystallizing beclomethasone in ultrasound having an intensity of 0.2 W/cm² or higher.

21. A method according to claim 20, comprising crystallizing beclomethasone in ultrasound having an intensity of 0.3 W/cm² or higher.

22. A method according to claim 15, wherein the crystallized beclomethasone particles have a size in the range from 0.5-10 microns.

23. A method according to claim 22, wherein the crystallized beclomethasone particles have a size in the range from 0.5-5 microns.

24. A method according to claim 22 wherein the crystallized beclomethasone particles have a size in the range of from 0.5-3 microns.

25. A method according to claim 15, comprising combining crystallized beclomethasone particles with a surfactant, under sterile conditions, to obtain a sterile nebulizer formulation of a volume from 1-3 ml.

26. A method according to claim 15, wherein the solvent is selected from acetone, ethanol, methanol and dichloromethane.

27. A nebulizer formulation obtained according to the method of claim 15.
